(19) United States
(12) Patent Application Publication
(10) Pub. No.: US 2018/0132902 A1
(43) Pub. Date: May 17, 2018

(54) UNIPLANAR PEDICLE SCREW

(71) Applicant: SHANDONG WEIGAO ORTHOPEDIC DEVICE COMPANY LTD, Weihai (CN)

(72) Inventors: Chunhui WU, Weihai (CN); Zheng WANG, Weihai (CN); Xuesong ZHANG, Weihai (CN); Ning XIE, Weihai (CN); Xiujun ZHENG, Weihai (CN); Yajun GAO, Weihai (CN)

(21) Appl. No.: 15/571,854

(22) PCT Filed: Apr. 29, 2016
(86) PCT No.: PCT/CN2016/080674

§ 371 Date: Nov. 6, 2017

(30) Foreign Application Priority Data
May 6, 2015 (CN) 201510225966.X

(51) Int. Cl. A61B 17/70

(52) U.S. Cl. CPC A61B 17/7037; A61B 2090/035; A61B 17/7032

(57) ABSTRACT

A uniplanar pedicle screw comprises a screw base, a positioning compression ring and a ball-head screw. Lateral bosses are provided at left and right sides of an upper portion of a ball head of the ball-head screw, and upper side surfaces of the lateral bosses are arc-shaped and project upwards. A central boss is provided on the ball head of the ball-head screw between the two lateral bosses, and an upper side surface of the central boss is arc-shaped and protrudes upwards. An arc center of the central boss and a connecting line of arc centers of the two lateral bosses are on the same straight line. Arc-shaped concave surfaces matching the two lateral bosses are provided at a lower portion of the positioning compression ring and at left and right sides of a rod accommodating groove.

### TECHNICAL FIELD

The present disclosure relates to the technical field of orthopedic medical equipment, and more particularly, to a uniplanar pedicle screw that has high overall strength and is not easily susceptible to deformation during use, allowing a screw-rod system with high overall stability, safety and reliability during surgical operation, and good fixing effect afterwards.

### BACKGROUND

As we all know, the existing pedicle screw types include uniaxial pedicle screw and polyaxial pedicle screw. When two or more vertebral bodies are being fixed by uniaxial pedicle screw, the orientation of the screw head is fixed and the two U-shaped slots of the screw heads are not aligned linearly after implanting one side of the pedicle screw, so that the connecting rod is difficult to implant. Although the polyaxial pedicle screw overcomes the above shortcomings, it is not suitable for spine orthopedic surgery due to its polyaxial characteristics. The advent of uniplanar pedicle screw solves the above technical problems. However, the structure of the existing uniplanar pedicle screw for achieving the single-plane swing of the screw body is implemented based on the polyaxial pedicle screw by changing the ball socket shape of the lower portion of the screw base and the ball head shape of the ball-head screw matching with the ball socket. For example, see the utility model patent No.: 201220532862.5 published on May 15, 2013, entitled “Uniplanar pedicle screw for minimally invasive reduction therapy”; and the utility model patent No.: 201220647516.1 published on Jun. 12, 2013, entitled “Expandable and pressurizable horizontal pendulum pedicle screw”, patent, both published by the State Intellectual Property Office of China.

### SUMMARY

### Technical Problem

The ball head and the ball socket matching with each other, once changed in shape, will have unbalanced stress therebetween, which affects the overall strength of the pedicle screw and thus is susceptible to deformation and screw failure during use in operation, rendering a serious influence on operation safety and a surgery risk for patients. Moreover, due to the small contact area between the ball head and the ball socket, the structure stability of the screw-rod system is poor and the ball head is easy to slip on the ball socket. As a result, the fixation angle of screw on patients is lost after the surgery, therefore affecting the surgical effect.

### Technical Solution

The object of the present disclosure aims to solve the above-mentioned deficiencies of the prior art and provides a uniplanar pedicle screw that has high overall strength and is not easily susceptible to deformation during use, allowing a screw-rod system with high overall stability, safety and reliability during surgical operation, and good fixing effect afterwards.

A uniplanar pedicle screw comprises a screw base, a positioning compression ring and a ball-head screw. The upper portion of the screw base is provided with a U-shaped slot for a connecting rod to go through. The screw base is provided with: an internal thread located on the inner wall at both sides of the U-shaped slot; a longitudinally through assembly hole provided in the screw base; a lower ball socket located inside the assembly hole and at the lower portion of the screw base, wherein the ball head of the ball-head screw is fitted in the screw base, matching with the lower ball socket. The positioning compression ring is disposed in the assembly hole and on an upper portion of the ball head of the ball-head screw, for pressing the ball head, and an upper portion of the positioning compression ring is provided with a U-shaped rod-containing groove. The upper portion of the ball head of the ball-head screw is provided on each of the left and right sides with a lateral boss which has an upper side surface being arc-shaped and protruding upwards. A a central boss is provided on the ball head of the ball-head screw between the two lateral bosses, and an upper side surface of the central boss is arc-shaped and protrudes upwards, connecting lines between an arc center of the central boss and arc centers of the two lateral bosses are in a same straight line. Two arc-shaped concave surfaces matching the two lateral bosses respectively are provided at a lower portion of the positioning compression ring on the left and right sides of the rod-containing groove. An arc-shaped recess matching the central boss is provided in the positioning compression ring between the two arc-shaped concave surfaces.

The outer wall of the positioning compression ring is provided with rotation-preventing lug on left and right sides of the rod-containing groove, while a rotation-preventing groove is provided on the inner wall of the screw base at a location corresponding to the rotation protrusion.

The screw base according to the present disclosure is provided with a snapping protrusion on the inner wall on both sides of the U-shaped slot under the internal thread on inner wall of the screw base. The upper end of the ring body of the positioning compression ring is peripherally provided with an outward protruded position-limiting flange matching with the snapping protrusion on each of the left and right sides of the rod-containing groove. The lower side surface of the position-limiting flange is an inclined plane. A rotation-preventing lug that protrudes outwardly is provided on the outer wall of the ring body on the lower side of the position-limiting flange A rotation-preventing slot is provided on the snapping protrusion and the inner wall of the screw base below the snapping protrusion. When the position-limiting flange fits beneath the snapping protrusion, the rotation-preventing lug fits into the rotation-preventing slot.

The connecting line between the arc center of the central boss and the arc centers of the two lateral bosses goes through the spherical center of the ball head of the ball-head screw. The arc radius of the central boss is 0.9 to 1.2 times, preferably 1.1 times, of the ball head radius of the ball-head screw, while the arc radius of the two lateral bosses is 0.4 to 0.8 times, preferably 0.5 to 0.6 times, of the ball head radius of the ball-head screw.

The outer periphery of the positioning compression ring according to the present disclosure has a column shape matching with the assembly hole.

### Advantageous Effects

The disclosure has the advantages of a uniform stress distribution, and a high overall strength. In addition, during use, the screw is not susceptible to deformation, and the screw-rod system thereof has a high overall stability, safety and reliability.

### BRIEF DESCRIPTION OF THE DRAWINGS

FIG. 1 is a schematic structural view of the present disclosure.

FIG. 2 is a 3D schematic structural view of the screw base according to the present disclosure.

FIG. 3 is a schematic structural view of the positioning compression ring according to present disclosure.

FIG. 4 is a right side view of FIG. 3.

FIG. 5 is a cross-sectional view taken along line A-A of FIG. 3.

FIG. 6 is a 3D schematic structural view of the positioning compression ring according to present disclosure.

FIG. 7 is a schematic structural view of the ball-head screw according to present disclosure.

FIG. 8 is a right side view of FIG. 7.

FIG. 9 is a 3D schematic assembly structural view of the ball-head screw according to present disclosure.

### DETAILED DESCRIPTION

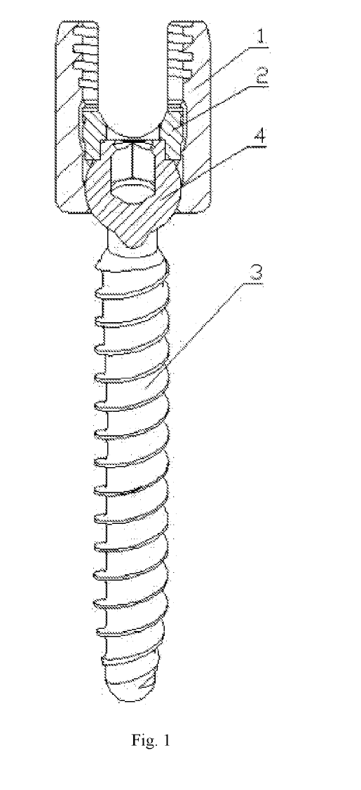

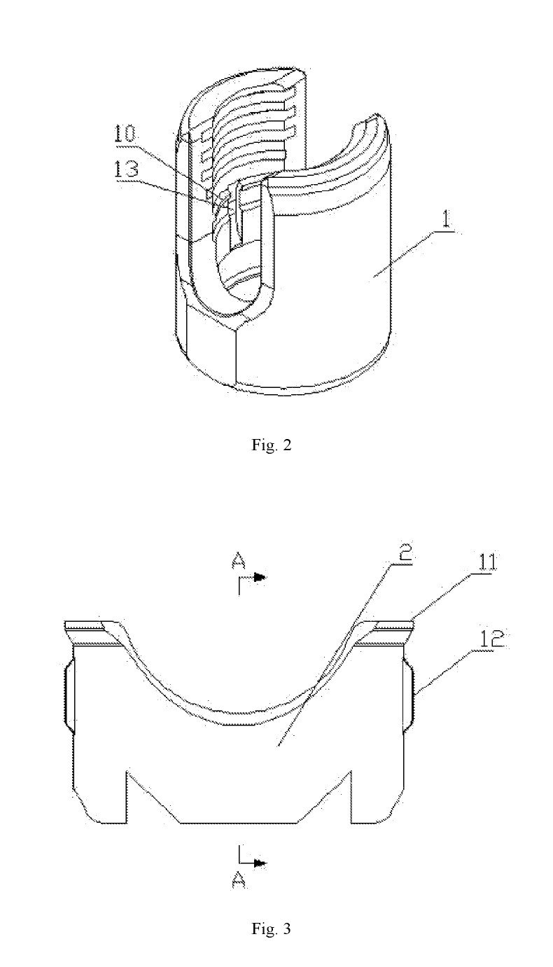

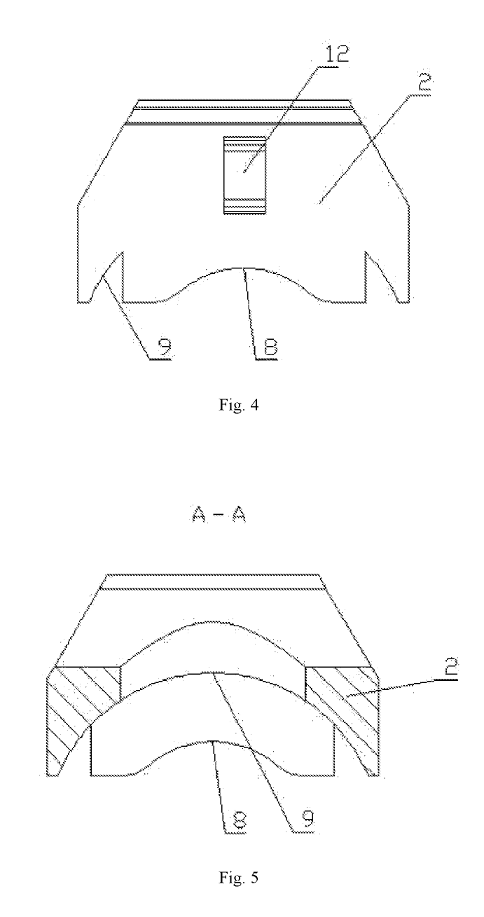

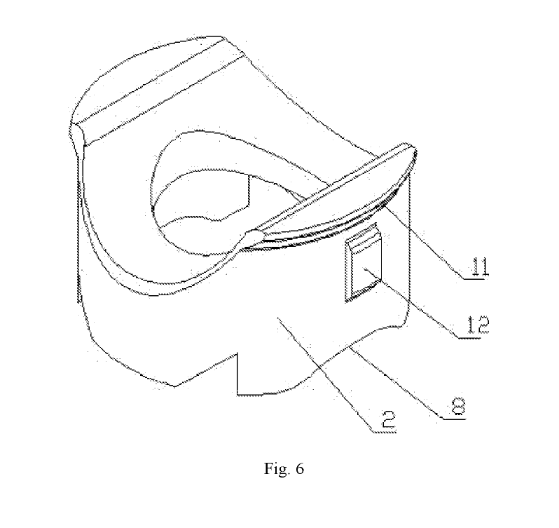

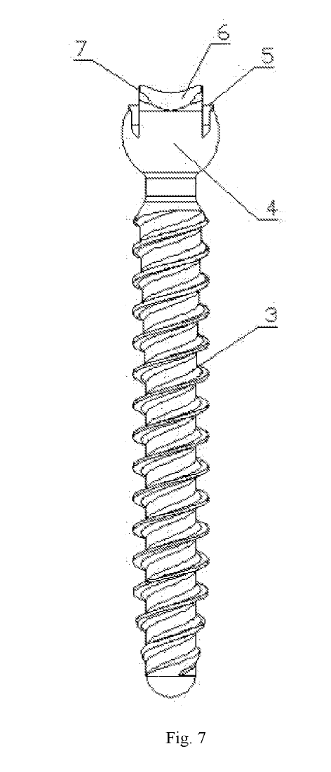

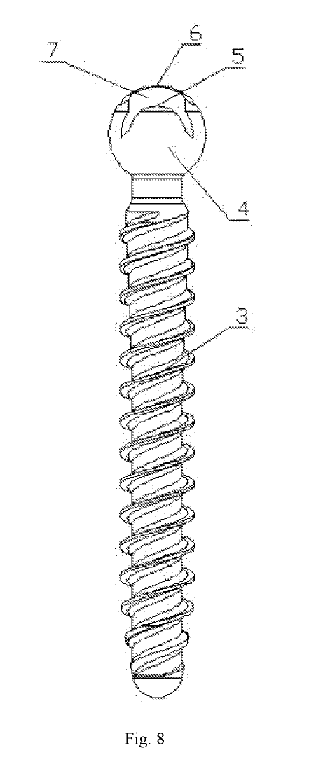

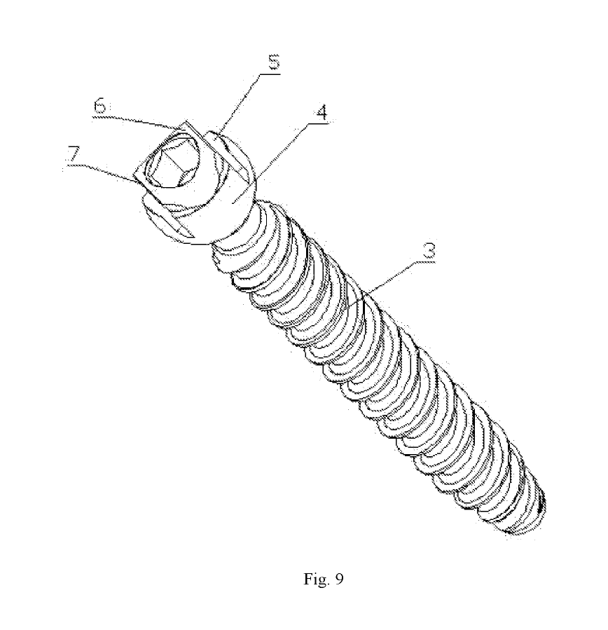

As shown in FIG. 1-10, a uniplanar pedicle screw comprises a screw base 1, a positioning compression ring 2 and a ball-head screw 3. The upper portion of the screw base 1 is provided with a U-shaped slot for a connecting rod to go through. The screw base 1 is provided with: an internal thread located on both sides of the U-shaped slot on the inner wall; a longitudinally through assembly hole inside; a lower ball socket inside the assembly hole on the lower portion of the screw base, wherein the ball head 4 of the ball-head screw 3 is fitted in the screw base 1, matching with the lower ball socket. The positioning compression ring 2 is disposed in the assembly hole and on an upper portion of the ball head 4 of the ball-head screw 3, for pressing the ball head 4, wherein the periphery of positioning compression ring has a column shape matching with the assembly hole. The upper portion of the positioning compression ring 2 is provided with a U-shaped rod-containing groove, wherein lateral bosses 5 are provided at the left and right sides of an upper portion of a ball head 4 of the ball-head screw 3. The upper side surface of the lateral bosses 5 is arc-shaped and projects upwards, and the spin axis of the arc surface on the upper side of the lateral boss 5 goes through the center of the ball head. A central boss 6 is provided on the ball head 4 of the ball-head screw between the two lateral bosses 5, and an upper side surface of the central boss 6 is arc-shaped and protrudes upwards. An arc center of the upper-side arc of the central boss 6 and a connecting line of the arc centers of the two lateral bosses 5 are in the same straight line. The arc radius of side surface of the central boss 6 is greater than the arc radius of the lateral boss 5. The two side surfaces 7 of the central boss 6 connecting with the two lateral bosses are vertical planes and parallel to the axis of the ball-head screw 3. The connecting line between the arc center of the central boss and the arc centers of both lateral bosses goes through the spherical center of the ball head of the ball-head screw. The arc radius of the central boss is 0.9 to 1.2 times, preferably 1.1 times, of the ball head radius of the ball-head screw, and the arc radius of the two lateral bosses is 0.4 to 0.8 times, preferably 0.5 to 0.6 times, of the ball head radius of the ball-head screw. Arc-shaped concave surfaces 8 matching the two lateral bosses 5 are provided respectively at a lower portion of the positioning compression ring 2 on the left and right sides of the rod-containing groove. An arc-shaped recess 9 matching the central boss 6 is provided in the positioning compression ring between the two arc-shaped concave surfaces 8. The connecting surface between the two arc-shaped concave surfaces 8 and the arc-shaped recess 9 are vertical planes.

Further improvements of the present disclosure may be as follows. The screw base 1 according to the present disclosure is provided with a snapping protrusion 10 on the inner wall on both sides of the U-shaped slot on the lower side of the internal thread on inner wall of the screw base. The upper end of the ring body of the positioning compression ring 2 is peripherally provided with an outward protruded position-limiting flange 11 matching with the snapping protrusion 10 on each of the left and right sides of the rod-containing groove. The lower side surface of the position-limiting flange 11 is an inclined plane. A rotation-preventing lug 12 that protrudes outwardly is provided on the outer wall of the ring body on the lower side of the position-limiting flange 11. A rotation-preventing slot 13 is provided on the snapping protrusion 10 and the inner wall of the screw base below the snapping protrusion. When the position-limiting flange fits beneath the snapping protrusion, the rotation-preventing lug 12 fits into the rotation-preventing slot 13.

### Industrial Utility

In use of the present disclosure, when the positioning compression ring engages the lower side of the snapping protrusion and presses against the ball head of the ball-head screw, the lateral bosses of the ball-head screw cooperate with the arc-shaped concave surfaces on both left and right sides of the rod-containing groove of the positioning compression ring, the central boss cooperates with the arc-shaped recess in the middle portion of the positioning compression ring, the two side surfaces of the positioning compression ring that are transiting from the two arc-shaped concave surfaces to the arc-shaped recess in the center portion of the ring adjoin and cooperate with the two side surfaces of the ball head transiting from the two lateral bosses to the central boss. The two side surfaces transiting from the two lateral bosses to the central boss on the ball head are parallel to the axis of the ball-head screw, so that the ball head is confined to only swing along the periphery of the arc-shaped concave surface. The present disclosure has a uniform stress distribution, a high overall strength, a safe and reliable intraoperative operation and a good fixation effect after operation.

1. A uniplanar pedicle screw, comprising a screw base, a positioning compression ring and a ball-head screw, wherein
an upper portion of the screw base is provided with a U-shaped slot for a connecting rod to go through;
the screw base is provided with: an internal thread located on an inner wall of the screw base, at both sides of the U-shaped slot; a longitudinally through assembly hole in the screw base; and a lower ball socket located in the assembly hole and at a lower portion of the screw base, wherein a ball head of the ball-head screw is fitted in the screw base, matching with the lower ball socket;
the positioning compression ring is disposed in the assembly hole and on an upper portion of the ball head of the ball-head screw, for pressing the ball head, and an upper portion of the positioning compression ring is provided with a U-shaped rod-containing groove;
the upper portion of the ball head of the ball-head screw is provided on each of left and right sides thereof with a lateral boss which has an upper side surface being arc-shaped and protruding upwards;
a central boss is provided on the ball head of the ball-head screw between the two lateral bosses, and an upper side surface of the central boss is arc-shaped and protrudes upwards, connecting lines between an arc center of the central boss and arc centers of the two lateral bosses are in a same straight line;
two arc-shaped concave surfaces respectively matching the two lateral bosses are provided at a lower portion of the positioning compression ring on left and right sides of the rod-containing groove; and
an arc-shaped recess matching the central boss is provided in the positioning compression ring between the two arc-shaped concave surfaces.

2. The uniplanar pedicle screw according to claim 1, wherein a rotation-preventing lug is provided on an outer wall of the positioning compression ring on each of the left and right sides of the rod-containing groove, while a rotation-preventing groove is provided on the inner wall of the screw base at a location corresponding to the rotation-preventing lug.

3. The uniplanar pedicle screw according to claim 1, wherein
the screw base is provided with a snapping protrusion on the inner wall thereof at both sides of the U-shaped slot below the internal thread on the inner wall of the screw base;
a position-limiting flange which protrudes outwardly and matches with the snapping protrusion is peripherally provided on an upper end of a ring body of the positioning compression ring on each of the left and right sides of the rod-containing groove, wherein a lower side surface of the position-limiting flange is an inclined plane;
a rotation-preventing lug that protrudes outwardly is provided on the outer wall of the ring body on the lower side of the position-limiting flange;
a rotation-preventing slot is provided on the snapping protrusion and the inner wall of the screw base below the snapping protrusion; and
when the position-limiting flange fits beneath the snapping protrusion, the rotation-preventing lug fits into the rotation-preventing slot.

4. (canceled)

5. The uniplanar pedicle screw according to claim 1, wherein
the connecting lines between the arc center of the central boss and the arc centers of the two lateral bosses go through a spherical center of the ball head of the ball-head screw;
an arc radius of the central boss is 0.9 to 1.2 times, preferably 1.1 times, of a radius of the ball head of the ball-head screw, while an arc radius of each of the two lateral bosses is 0.4 to 0.8 times, preferably 0.5 to 0.6 times, of the radius of the ball head of the ball-head screw.

6. The uniplanar pedicle screw according to claim 4, wherein an outer periphery of the positioning compression ring has a column shape matching with the assembly hole.
